(19) United States
(12) Patent Application Publication
(10) Pub. No.: US 2006/0213514 A1
(43) Pub. Date: Sep. 28, 2006

(54) Delivery device for a powder aerosol

(76) Inventors: Robert Price, Chepstow (GB); John Nicholas Staniforth, Bath (GB); Derek Alan Woodcock, Berkhampstead (GB); Paul Michael Young, Bath (GB); James A. Byrnes, Leawood, KS (US); J. Nita Cogburn, Overland Park, KS (US); Douglas R. Dockhorn, Stilwell, KS (US); Robert J. Dockhorn, Prairie Village, KS (US); Edgar W. Mitchell, Overland Park, KS (US); Susan J. Prather, Kansas City, MO (US)

Correspondence Address: POLSINELLI SHALTON WELTE SUELTHAUS P.C., 700 W. 47TH STREET, SUITE 1000, KANSAS CITY, MO, 64112-1802, US

(21) Appl. No.: 11/296,637

(22) Filed: Dec. 7, 2005

Related U.S. Application Data
(63) Continuation-in-part of application No. PCT/GB04/02490, filed on Jun. 14, 2004.

(30) Foreign Application Priority Data
Jun. 12, 2003 (GB) GB0313604.1

(51) Int. Cl. A61M 15/00; A61M 16/00

(52) U.S. Cl. 128/203.15

(57) ABSTRACT

A delivery device for a medicament including: a housing, a receptacle holding a medicament in the form of a power; and a source of propellant, wherein the housing provides an inlet and an outlet for the receptacle, wherein the inlet is in fluid communication with the source of propellant and is directed against the medicament and the outlet is spaced from the medicament to allow aerosolization of the medicament; the device provides improved delivery efficiency, particularly a delivered fine particle fraction of greater than 20% by weight.

This Application is a continuation-in-part of U.S. Non-Provisional Application No. ______ filed on Dec. 6, 2005 which is the National Stage Application of PCT/GB2004/002490 having an international filing date of Jun. 14, 2004, which claims priority to Great Britain Patent Application No. 0313604.1 filed Jun. 12, 2003.

### FIELD OF THE INVENTION

The present invention relates to a hand-held delivery device for a medicament in the form of a powder, typically as an aerosol of powder particles. In particular, the delivery device may be used for delivery of a medicament without a carrier into the airways/lungs.

### BACKGROUND OF THE INVENTION

Two main types of hand held devices for delivering doses of aerosol medicament to a patient are known. These are a propellant-driven metered dose inhaler (MDI) and a dry powder inhaler (DPI).

In an MDI, the medicament is suspended or dissolved in a propellant. The propellant is provided in a pressurized canister having a metered valve which, upon activation, produces a single dose of the medicament in the form of a gas stream. The device may include a tapered discharge nozzle baffle or a venturi to accelerate particles through a discharge nozzle, and to remove oversized particles. Suitable halocarbons used in an MDI include hydrofluorocarbons, hydrofluorochlorocarbons and fluorochlorocarbons having a low boiling point, such as those marketed under the trade mark “Freon”.

The problem with the MDI device is that when it is used to deliver a medicament to a patient's lungs, only a small percentage of the medicament is delivered in a respirable form (approximately 8 weight % fine particle fraction). This is because the high linear speed at which the dosage leaves the device combined with incomplete evaporation of the propellant causes much of the medicament to impact and stick to the back of the throat, causing localized problems in the impact area. This medicament is generally later swallowed by the patient which, for some medicaments such as bronchodilators, can lead to unwanted systemic side effects.

A further problem is that MDIs require coordination between activation and inhalation. Many patients are incapable of this, especially infants, small children and the elderly.

In an attempt to overcome this problem, MDIs have been used with a “spacer” which provides an additional volume in which the propellant may evaporate. It has been found that the fine particle fraction is deposited within the spacer instead of the back of the patient's throat.

In a DPI device, no propellant is used but instead the device relies upon a burst of inspired air drawn through the unit by the patient. These devices suffer from the problem that the force of inspiration varies considerably from person to person. Some patients, particular those with lung problems whom such devices are designed to treat, are unable to generate sufficient air in-flow to activate the device. DPIs have many of the disadvantages of MDIs because of incomplete particle dispersion and the impact at the back of the throat.

In an attempt to overcome this problem with DPIs, the medicament for use in such devices has been formulated in a particular way to aid de-agglomeration. Thus the medicament is generally provided with a carrier or is processed in such a way that weakly bound agglomerations of the medicament are produced which the device may more easily break up. Therefore DPIs are unsuitable for use with medicaments which, due to their high dosage rate, cannot be administered with a carrier or which cannot be further processed in this way. Formulated DPIs where the medicament is administered with a carrier have a problem that the amount of administered medicament in a respirable form is low because the medicament remains adhered to the carrier.

There are other medicaments such as pumactant which is a blend of dipalmitoylphosphatidylcholine (DPPC) and phosphatidylglycerol (PG) (DPPC:PG 7:3), which is very cohesive due to its low particle size, high moisture affinity and predominantly amorphous structure. A device suitable for administering this medicament to the lungs of a patient is needed.

A way of ameliorating these problems has been sought.

### SUMMARY OF INVENTION

In one aspect, the present invention provides a delivery device for a powdered medicament comprising: a housing, a receptacle holding a medicament in the form of a powder, and a source of propellant, wherein the housing has an inlet for the receptacle in fluid communication with the source of propellant and an outlet for the medicament, wherein the inlet is directed against the medicament and the outlet is spaced from the medicament to allow aerosolization of the medicament.

A surprising advantage of the device according to the present invention is that it has much greater efficiency than known inhaler devices. It has been found that the device efficiency is about 70.1 weight % in terms of the weight of the delivered dose compared to the weight of the dose loaded in the device (as measured using a Marple Miller impactor; the data is shown in Example 2 below). In particular, the delivered fine particle fraction is at least 20 weight % of the amount of medicament originally loaded in the receptacle. Where the device has been optimized, a delivered fine particle fraction of 40 weight % has been achieved.

The advantages of the spaced arrangement of the outlet (which is the feature that the outlet is spaced from the medicament to allow aerosolization of the medicament) include that it overcomes the problems of incomplete evaporation of the propellant (where the propellant is liquefied gas) and patient coordination. The problem with patient coordination is improved because there is a slight delay between activation of the device and delivery of the aerosolized medicament from the outlet for the device according to the invention particularly compared to a standard MDI. This is because the aerosol is first generated in the receptacle and then has to pass through the outlet before reaching a patient. This is advantageous because a patient normally finds it difficult to simultaneously activate an inhaler and inhale; it is easier to activate the inhaler and then inhale which the device according to the present invention would allow.

The inlet is generally in fluid communication with the source of propellant such that there is a propellant pathway from the source to the inlet. The propellant pathway is preferably provided with at least one choke to decelerate the propellant. The propellant pathway choke may be in the form of a constriction or a baffle; preferably it is in the form of a constriction. A propellant pathway choke is useful where the medicament is at least partially amorphous such that it is vulnerable to becoming waxy or being compressed when the propellant is directed against it. This would clearly be disadvantageous because an aerosol of the medicament would be generated less efficiently, if at all.

The propellant pathway generally passes from the source of propellant through the housing and then through the header unit to the inlet. It is optionally either formed by the housing or is in the form of tubing, especially medical grade tubing.

The inlet is preferably in the form of an inlet tube. The inlet tube is in fluid communication with the propellant pathway and leads from the housing and is directed against the medicament. The inlet preferably has an end which is directed against the medicament. The end of the inlet is preferably in the form of a flared tube or of a “shower-head” such as a flared and perforated end. The inlet tube preferably extends into the receptacle.

Where it is said that the inlet is directed against the medicament, it should be understood that the inlet is either adjacent to the medicament such that there is a gap between the inlet and the medicament or the inlet is in contact with the medicament. Where the inlet is in contact with the medicament, it is optionally either touching the medicament or inserted into the medicament.

In addition to or as an alternative to a propellant pathway choke, the inlet, particularly the inlet tube, is preferably provided with one or more perforations. Such a perforation is useful as an alternative to a propellant pathway choke as it would decelerate the propellant exiting the inlet before it is directed against the medicament. Furthermore, a perforation in the inlet may also be useful in assisting in the formation of the aerosol of medicament.

In a preferred form of the device according to the invention, the spaced arrangement of the outlet and/or the propellant pathway choke (if present) are preferably arranged such that on activation of the device, a stable aerosol of the medicament is formed in the spaced arrangement. Such a stable aerosol of the medicament will be referred to herein as a standing cloud of medicament.

A device arranged to produce a standing cloud of medicament is particularly advantageous because it makes the medicament easier to administer. Such a device preferably has a normally sealed outlet. Preferably the outlet has an outlet pathway which connects to the exterior of the device (the outlet is in fluid communication with the outlet pathway); more preferably the outlet pathway ends in an exterior outlet; most preferably, the exterior outlet is normally sealed. Such an arrangement is advantageous in terms of patient compliance because a patient is then able to first activate the device to generate the standing cloud of medicament and then open the normally sealed outlet (especially the normally sealed exterior outlet) to inhale the medicament thus avoiding any problem with coordinating activation with inhalation.

The receptacle generally has a bottom containing the medicament and a top which connects to the housing. The outlet is preferably arranged to open into the receptacle at the top of the receptacle. Preferably the outlet is formed as a hole in the housing which is in fluid communication with an outlet pathway to the exterior of the housing.

The source of propellant may optionally be provided by a canister of gas (e.g., compressed gas or liquefied gas) or by a supply of compressed gas such as a supply line of compressed gas such as that typically provided in a hospital room.

The device of the invention is preferably a handheld device using a canister of a pressurized gas as the source of propellant.

The device according to the invention is optionally provided with a mouthpiece attached to the outlet to aid self-administration of the medicament by a patient. Any known mouthpiece may be used in association with the device according to the invention.

Alternatively, the outlet may be provided with a tube for engaging with a breathing tube for a patient using a respirator to enable a third party, e.g., a healthcare professional such as a doctor or nurse to administer a medicament to the patient.

The device has been shown (in Examples 1 and 2) to be highly effective for aerosolizing even highly cohesive powders, such as pumactant. As a result of the high energy transfer, the device also provides a high respirable fraction in the delivered powder and a high delivered dose relative to the loaded dose. Accordingly it provides a vehicle for dispensing powders that hitherto have required formulation with large quantities of excipients, such as lactose, for aerosolization. This causes problems of bulk when high doses of active are needed. The present invention thus allows active materials that require high doses to be delivered in respirable “drug only” form, i.e., without a carrier.

The outlet of the header unit is generally in fluid communication with the exterior of the housing and may be in the form of a passage formed in the header unit or in the form of tubing, especially medical grade tubing. The outlet is preferably provided with one or more chokes for decelerating the aerosol of the medicament where the device is not a device arranged to produce a standing cloud of medicament. Having one or more outlet chokes is useful because it increases the delay between activation of the device and delivery of the medicament, aiding patient compliance. It is also useful because it reduces the problems of reduction in delivered respirable dose because of impact at the back of a patient's throat.

The one or more outlet chokes are preferably one or more constrictions and/or one or more baffles in the outlet. A constriction for use as a choke in the present invention is preferably a reduction in the cross-section of the propellant pathway and/or of the outlet. The reduction in cross-section is optionally either temporary such that after the choke, the propellant pathway and/or outlet revert to their previous cross-section or it is permanent. A baffle for use as a choke in the present invention is preferably provided as an abrupt change in direction of the propellant pathway and/or of the outlet such as a change of direction of from 45 to 135 degrees (measured as the angle between the outlet or propellant pathway before and after the baffle), especially a change of direction of about 90 degrees.

Accordingly, in a further aspect the present invention provides a method of dispensing a medicament as an aerosol to a patient in need of such treatment which method comprises the steps of: providing a receptacle having an opening which receptacle contains the medicament in powder form; discharging a pressurized propellant from a canister or cartridge through a delivery tube extending into the receptacle and directed at the medicament so as to fluidize it; forming an aerosol by transfer of energy from the propellant to the powder; and discharging the aerosol through an outlet passage provided at the opening of the receptacle.

Where the source of propellant is a removable gas canister and the receptacle is removable, the device may be provided in the form of a first kit according to the invention which kit comprises a gas canister, a receptacle containing a medicament in powder form and a first delivery device housing including the header unit.

Therefore according to the invention, there is provided a first delivery device housing suitable for use in a first kit according to the invention having a first and a second open-ended compartment wherein the first compartment is adapted to receive a source of propellant and the second compartment is adapted to receive a receptacle containing a medicament in powder form wherein the second compartment provides an inlet for propellant in fluid communication with the first compartment and an outlet wherein the outlet, in use, is spaced from the medicament to allow aerosolization of the medicament.

The first kit optionally further comprises a closure (such as an end cap) for sealing the receptacle in the second compartment.

Alternatively, the receptacle may be provided in association with the header unit such that a second kit according to the invention comprises a source of propellant, a dispensing receptacle according to the invention and a second delivery device housing according to the invention.

The dispensing receptacle according to the invention comprises a receptacle containing a medicament unit in fluid tight engagement with a header unit wherein the header unit provides the receptacle with an inlet for propellant and an outlet wherein the outlet is spaced from the medicament to allow aerosolization of the medicament in use and wherein the header unit has a propellant entry connector in fluid communication with the inlet for propellant.

The second delivery device housing according to the invention has a first open-ended compartment which is adapted to receive a source of propellant and a clip which is adapted to receive a dispensing receptacle according to the invention wherein the clip has a propellant connector associated with it which exit connector is arranged to engage with the entry connector of the dispensing receptacle.

A first kit according to the invention preferably comprises a plurality of receptacles. Optionally in the first kit, the receptacle and source of propellant may be provided in the form of combined supply for the first delivery device housing such that the receptacle and source of propellant are linked for combined insertion into the housing.

The receptacle containing the medicament can be any suitable packaging container, for example, a glass or plastic vial or a blister pack. Typically the opening of the receptacle is sealed to preserve sterility of the powder and avoid water adsorption. After removal of the seal the receptacle may then inserted into the device according to the invention such that the opening of the receptacle is brought into a fluid-tight engagement with the housing, preferably via a gasket or sealing ring. The receptacle may be held in engagement with the housing by a screw or twist connection. Alternatively a clamp on the housing or a closure (such as an end cap) for the other end of the compartment may be provided to support the receptacle and to press the opening of the receptacle against the housing or gasket, if present. The receptacle may contain a single dose of powder for one-time use, or sufficient powder for several doses. The medicament is preferably in the form of a respirable powder. More preferably the medicament is in the form of a powder having a mass median aerodynamic diameter (MMAD) measured by laser diffraction of less than 20 μm, preferably less than 10 μm, more preferably less than 5 μm, most preferably from 1 μm to 5 μm.

Where the receptacle is a vial, the spaced arrangement of the outlet is provided by the vial. This is because there is typically empty space between the contents of the vial and its opening. For a 10 ml vial, the volume of the contents is usually from 0.5 to 2 ml, leaving an empty volume of 8 to 9.5 ml. If the outlet of the device of the invention is formed in the header unit, this empty volume has been found to be sufficient to provide the spaced arrangement between the medicament and the outlet for certain medicaments, particularly pumactant.

Where a blister pack is used as the receptacle, the device preferably comprises an open-ended compartment for receiving the blister pack. The volume of the open-ended compartment preferably provides the spaced arrangement for the outlet. This is because in a blister pack there is usually insufficient volume between the opening of the blister pack and the medicament for this volume to be used as the volume for the spaced arrangement of the outlet.

This volume of the spaced arrangement of the outlet is preferably chosen according to the amount of medicament to be aerosolized and its degree of cohesion. It is preferably not so small that the medicament cannot be aerosolized. Also it is preferably not so large that the aerosol of the medicament is dissipated and destabilizes.

The source of propellant is generally arranged in fluid-tight engagement with the propellant pathway by a screw, twist or push connection. Where the source of propellant is a gas canister, it is preferably a replaceable canister with a metering valve having an extended valve stem which is pressed to discharge gas.

The device is preferably arranged such that in use the valve is above the canister. This is advantageous because a patient can then use a thumb to activate the canister by pressing on its base. When using an MDI, the patient is instructed to use a finger to activate it. As substantial pressure can be required to activate a metered valve, this arrangement can lead to compliance problems which the present invention overcomes.

The device of the invention can be used to administer any medicament suitable for administration by inhalation such as a SAPL (surface active phospholipid) composition, such as pumactant, a bronchodilator or a steroid.

The propellant used in the present invention is preferably carbon dioxide, nitrogen, air, or a halocarbon (e.g., a fluorocarbon such as HFA-134a or HFC-227).

### BRIEF DESCRIPTION OF THE DRAWINGS

The invention is illustrated by way of example by the Figures of the accompanying drawings in which:

FIG. 1 is a cross-sectional view of a first embodiment of a device according to the invention;

FIG. 1a is a plan view of the device shown in FIG. 1;

FIG. 1b is a perspective view of a part of the device shown in FIG. 1;

FIG. 2 is a cross-sectional view of a second embodiment of a device according to the invention;

FIG. 3 is a cross-sectional view of a third embodiment of a device according to the invention;

FIG. 4 is a cross-sectional view of a first embodiment of a kit according to the invention;

FIG. 5 is a cross-sectional view of a second embodiment of a kit according to the invention;

FIG. 6 shows a graph illustrating the data obtained from an in-vitro assessment of Pumactant aerosolized and delivered by a device according to the invention using a 1.5 m long 1 mm diameter endotracheal tube;

FIG. 7 shows the relationship between loaded dose and delivered dose in the procedure of Example 2; and

FIG. 8 charts fine particle fractions as a function of canister pressure in the procedure of Example 2.

FIG. 9 is a side view of a fourth embodiment of a device according to the invention.

FIG. 10 is a front view of the fourth embodiment of a device according to the invention.

FIG. 11a is a top view of a fourth embodiment of a device according to the invention.

FIG. 11b is a bottom view of a fourth embodiment of a device according to the invention.

FIG. 12 is a cross-sectional view of a fourth embodiment of a device according to the invention.

FIG. 13 is a further cross-sectional view of a fourth embodiment of a device according to the invention.

FIG. 14 is a front view focusing on the mouthpiece of a fourth embodiment of a device according to the invention.

FIG. 15 is a view of a bulkhead of a fourth embodiment of a device according to the invention.

FIG. 16 shows the delivery characteristic of aerosols from a fourth embodiment of a device according to the invention.

### DETAILED DESCRIPTION OF PREFERRED EMBODIMENTS

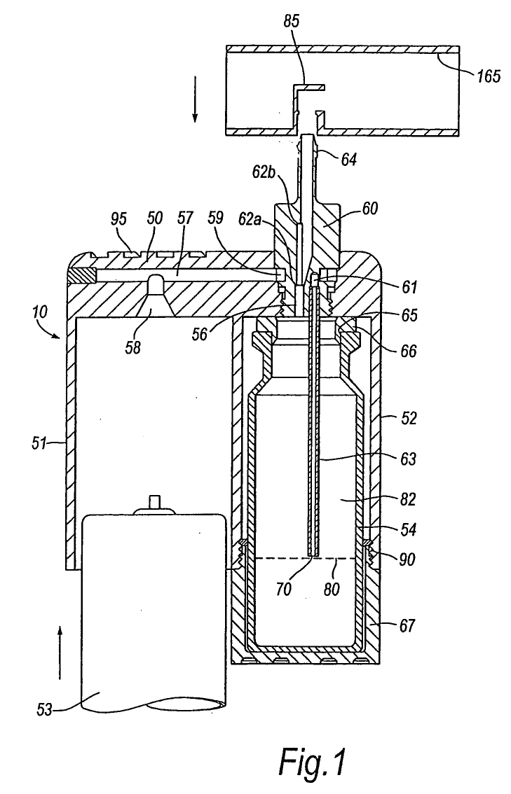

A first embodiment of a dispenser device 10 of this invention is shown in FIGS. 1, 1a and 1b of the accompanying drawings. This embodiment has a housing 50 in the form of two open-ended cylinders 51, 52 mounted side by side and forming respective chambers to hold a canister of pressurized propellant 53 (shown in part) and a receptacle 54 of medicament in powder form. The upper surface 95 of the housing 50 is moulded to provide a ridge surface to aid a patient's grip on the device.

A propellant pathway 57 is provided through the housing 50. The propellant pathway 57 links a propellant inlet fitting 58 for propellant formed at the top end of cylinder 51 and an aperture 59 formed in the end portion 56. Aperture 59 has a smaller cross-section than that of the propellant pathway 57 such that it provides a propellant pathway choke to decelerate fluid flow through the propellant pathway 57. In an alternative embodiment, the propellant pathway choke is in the form of a baffle.

The aperture 59 is adjacent to a screw-in header unit 60 seen in more detail in FIG. 1b. The header unit 60 has a circumferential groove 68. The housing 50 and header unit 60 are arranged such that the passageway 57 meets the circumferential groove 68. The groove 68 provides a further propellant pathway choke which is in the form of a baffle. The header unit 60 has an inlet pathway 61 which exits the base of the header unit 60. The direction of the inlet pathway 61 is at an angle of approximately 90 degrees to the propellant pathway 57. Thus where groove 68 and the inlet pathway 61 meet, a further propellant pathway choke is provided in the form of a baffle.

In an alternative embodiment, the header unit 60 is integrally moulded with the housing 50 such that the features of the header unit 60 are provided by the housing itself.

An inlet tube 63 is inserted into the pathway 61 in the base of the header unit 60 and extends into the interior of the cylinder 52. Thus the inlet tube 63 extends into the receptacle 54. An outlet 55 is also formed as a hole in the base of the header unit 60. Outlet 55 does not extend into the receptacle 54. Outlet 55 is spaced from the opening 65 of the receptacle 54 by a gasket 66 which seals the receptacle. In an alternative embodiment, outlet 55 is substantially flush with the opening 65 of receptacle 54. Outlet 55 is in fluid communication with outlet pathway 56 which extends to an outlet port 64 on the outer surface of the header unit 60.

Outlet pathway 56 is provided with a constriction 62a where the cross-section of outlet pathway 56 is reduced. Outlet pathway 56 is also provided with a baffle 62b. Constriction 62a and baffle 62b are arranged to decelerate fluid flow through outlet pathway 56.

The base of the header unit 60 is provided with a gasket 66 which provides a fluid-tight seal between the header unit 60 and receptacle 54. The receptacle 54 is held tightly against gasket 66 because the open-end of cylinder 52 is sealed by screw-threaded end cap 67.

The propellant canister 53 is provided as a replaceable unit, and most suitably contains a compressed gas as propellant, such as carbon dioxide, nitrogen or air. However other conventional propellants, such as a low boiling liquid, preferably a fluorocarbon such as HFA-134a or HFC-227, under sufficient pressure to maintain the propellant liquid at normal room temperature, may also be used. The propellant canister 53 is a conventional unit which has a metering valve with a protruding valve stem, which when depressed releases propellant through a passage way in the valve stem. In use of the device, the canister 53 is inserted into the cylinder 51 so that the valve stem is located in gas inlet fitting 58. The fitting 58 is dimensioned so that the valve stem is a press fit in the fitting 58 and so holds the canister 53 in the interior of the cylinder 51.

The receptacle 54 containing medicament, is typically supplied as a sealed unit. Receptacle 54 has an opening 65 which before use is sealed to protect the powder contents. After stripping the seal, the receptacle 54 is introduced into the interior of the cylinder 52, so that the opening 65 is forced against a resilient gasket 66 and the delivery tube 63 enters into the receptacle 54. The open end of the cylinder 52 is closed with an end cap 67 which engages with the cylinder 52 by a mutual screw-thread 90. The end cap 67 provides the means by which the receptacle 54 is maintained in position with the opening 65 sealingly engaged with the gasket 66.

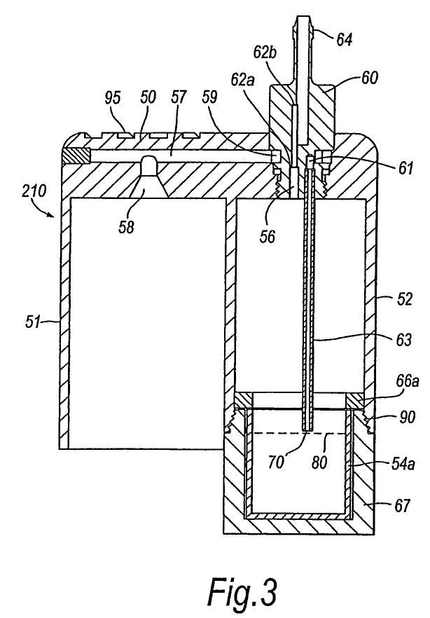

An alternative arrangement to FIG. 1 is shown in FIG. 3. This device 210 is the third embodiment of the device according to the invention. Like reference numerals are used to represent like features of the first embodiment. In this embodiment, receptacle 54a is smaller than the receptacle 54 shown in FIG. 1. This receptacle 54a is in the form of a blister pack. Here there is a much smaller gap between the opening 65 of the receptacle 54 and the medicament level 80. A gasket 66a is provided adjacent to screw-thread 90. This is in order that in use, a blister pack 54a can be placed into end cap 67 which is then used to close cylinder 52. The mouth 65 of blister pack 54a then engages with gasket 66a which holds the blister pack in place. The outlet tip is directed against the medicament in the blister pack 54a. The device 210 works in the same way as the device 10 according to the first embodiment of the invention.

To use the device, the user pushes the end of the gas canister 53 into the interior of the cylinder 51. As the valve stem of the canister remains secured in the passage 58, the inward movement of the canister effectively depresses the valve stem, and releases propellant through the valve stem into the passageway 57. The propellant proceeds through aperture 59, circumferential groove 68, inlet passage 61 and into the receptacle 54 via delivery tube 63. The delivery tube 63 is dimensioned so that its outlet tip 70 is directed at or dipping into the powder contents of the receptacle 54, so that the propellant is directed against the powder. (To avoid damage when the closure 67 is removed and no receptacle 54 is loaded, the tube 63 is dimensioned so that the tip 70 lies within the cylinder 52). As a result, the propellant fluidizes the powder and forms a respirable aerosol in the volume 82 between the level of the medicament 80 and the outlet 55. The aerosol exits the receptacle 54 via the outlet 55 and the outlet passage 56. On its way through the outlet passage, the aerosol is decelerated by constriction 62a and baffle 62b.

The outlet port 64 may be formed as, or exit into, a mouthpiece 165 or a shaped end piece which is a comfortable shape to place in the mouth, nose or other body orifice of a patient. The mouthpiece 165 shown has a baffle 85. Alternatively the outlet 64 may be extended to form, or connect to, a respiration tube, e.g., a tracheal tube (not shown).

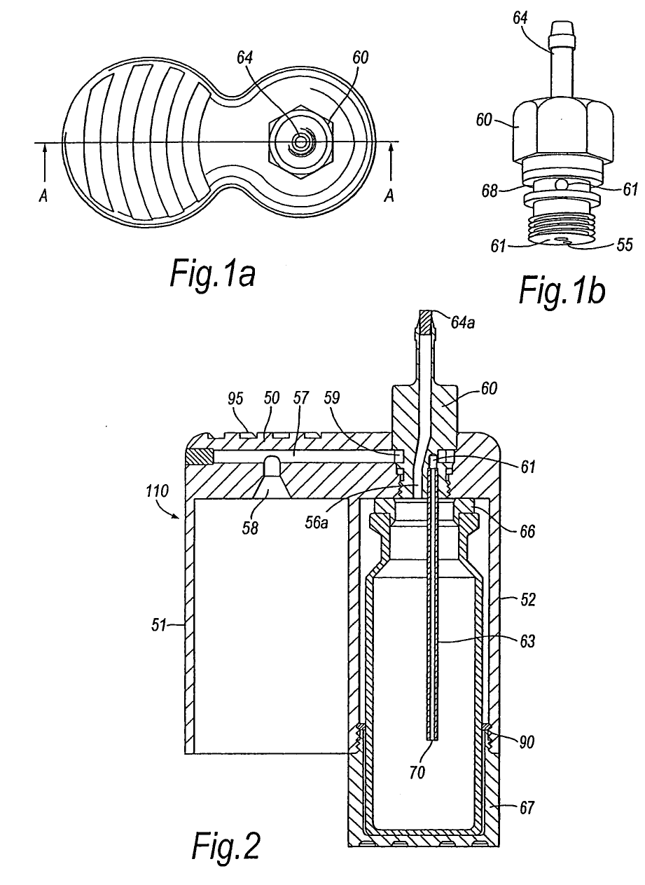

A second embodiment of a dispenser device 110 according to the invention is shown in FIG. 2. Like reference numerals are used to represent like features of the first embodiment. The device 110 differs from device 10 in that outlet pathway 56a lacks the constriction 62a and baffle 62b of the first embodiment. The device 110 also differs in that outlet port is sealed with removable seal 64a. The device 110 works in the same way as device 10 except that it is suitable for optimization to generate a stable aerosol or standing cloud on activation.

As an alternative in device 110, removable seal 64a is replaced by a normal outlet port 64.

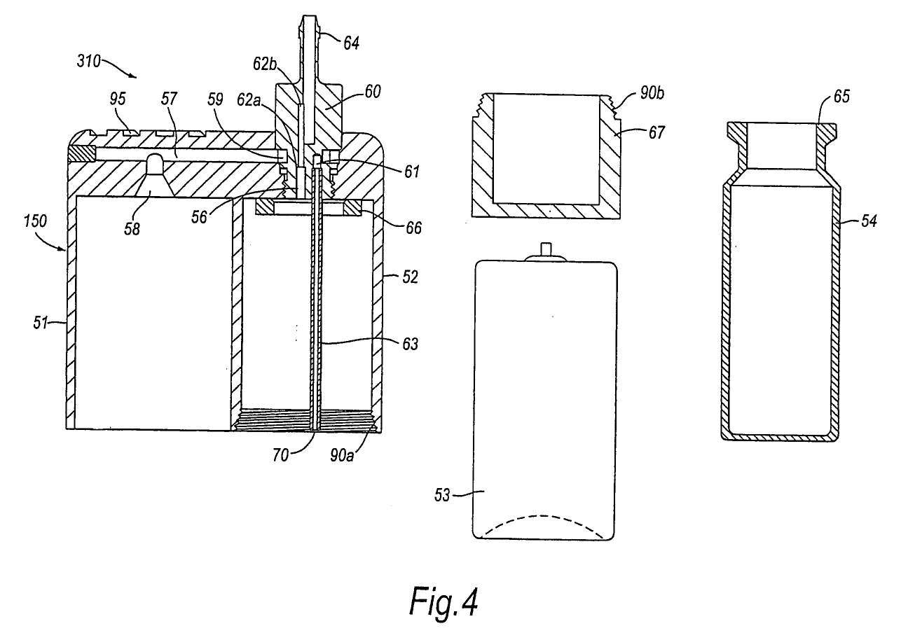

A first embodiment of a kit 310 according to the invention is shown in FIG. 4. Kit 310 has a device housing 150, an end cap 67, a source of propellant 53 and a receptacle 54. Like reference numerals are used to represent like features of the first embodiment.

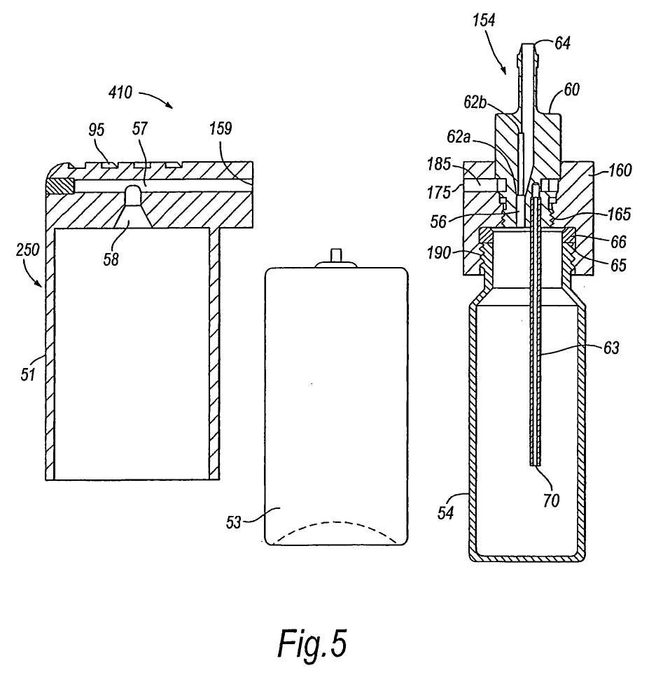

A second embodiment of a kit 410 according to the invention is shown in FIG. 5. Kit 410 has a device housing 250, a source of propellant 53 and a dispensing receptacle 154. Like reference numerals are used to represent like features of the first embodiment.

Device housing 250 has a propellant exit connector 159 which is provided with a constriction to act as a propellant pathway choke. Device housing 250 also has a clip (not shown) for engaging dispensing receptacle 154.

Dispensing receptacle 154 has a receptacle connector 160, header unit 60 and receptacle 54. Receptacle connector 160 joins header unit 60 to receptacle 54. Header unit 60 engages with the receptacle connector 160 by screw fitting 165 and receptacle 54 engages with the receptacle connector 160 by screw fitting 190. Receptacle connector 160 has a propellant entry connector 175 which is in fluid communication with a propellant pathway 185 which leads to circumferential groove 68 on the header unit 60.

To use the kit according to the second embodiment, the dispensing receptacle 154 is clipped onto the device housing 250 such that the propellant exit connector 159 of the device housing 250 engages with the propellant entry connector 175 of the receptacle connector 160. The assembled kit then functions in the same way as the device 10 according to the first embodiment of the invention.

The efficacy of the device according to the invention is illustrated in the following Examples:

### EXAMPLE 1

A device according to the invention has been successfully used in experimental veterinary treatment of respiratory disorders in horses using pumactant, as detailed below.

Horses are susceptible to a plethora of respiratory complaints. Heaves is the equine equivalent of asthma and both diseases share similar etiology and pathology. The disease, in the equid, has been shown to proceed via a Th2 cytokine driven mechanism (Lavoie, J-P., Maghni, K., Desnoyers, M., Taha, R., Martin, J. G., and Hamid Q. A. (2001) Neutrophilic airway inflammation in horses with heaves is characterized by a Th2 cytokine profile. Am. J. Respir. Crit. Care. Med 164 1410-1413). They, like their human counterparts, have poor compliance and a massive lung surface area estimated to be in the region of 1000 m².

The aim of the study was to investigate the use and approach to delivery of a thermally labile, hygroscopic and dry surfactant, ensuring an acceptable physicochemical character. The surfactant used was pumactant, (formerly known as ALEC), which is a mixture of two phospholipids: DPPC and PG in a ratio of 7 parts:3 parts DPPC:PG. This specific ratio of phospholipids has a low phase transition temperature (approximately 32° C.) which it is believed facilitates rapid spreading at body temperature when in contact with an air/water interface. It is also highly rich in DPPC which mimics the high percentage of endogenous DPPC in vivo.

It was used as a dry powder because in a previous human (allergic asthma) study (Babu, K. S., Woodcock, D. A., Smith, S. E., Heminsley, A. M., Little, L., Staniforth, J. N., Holgate, S. T., and Conway, J. H. Pumactant abolishes early asthmatic response in patients with allergic asthma, Presentation given at the American Thoracic Society, Atlanta, USA (2002)), the preparation had been delivered as a dry powder and produced excellent clinical results. Currently, surfactants are delivered as aqueous based preparations; however, it is has been demonstrated that surface activity is reduced when the active is delivered as an aqueous suspension. Indeed, delivery of aqueous preparations is counterintuitive in certain disease states: RDS.

Pumactant is physically unstable even at conditions of low relative humidity (approximately 30%), and it can undergo morphological changes, which may affect particle size. Careful attention must therefore be applied to storage and delivery of the surfactant.

The device according to the present invention was used for delivery of pumactant because it has the following advantageous delivery characteristics:

The use of a particulate free and low moisture gas source

Capable of aerosolizing and de-aggregating large particles

Adaptation to equine anatomy and physiology

Ease of use for clinician/veterinarian

The pumactant was administered by utilizing an endotracheal tube, bypassing the nasal anatomy, delivering the material to each bronchus; this arrangement, obviously, would also omit patient compliance issues.

The use of an equine model, as previously described, facilitated the delivery of a mass of powder not conventionally delivered to the respiratory tract. The device and mode of delivery is erstwhile described, but what is not apparent is the particle size distribution of the material used. Since it was manufactured as freeze dried powder the particle size distribution does not conform to a conventional respiratory particle size distribution. In fact, the MMAD (mass median aerodynamic diameter) as evaluated by laser diffraction was approximately 10 microns with a distribution that ranged from approximately 1 to 180 microns. This was initially a concern. Current practice delivers particles in a 2-5 MMAD micron range and, whilst direct delivery to each bronchus removed some proximal deposition, it had not been established the extent to which a large particle would penetrate.

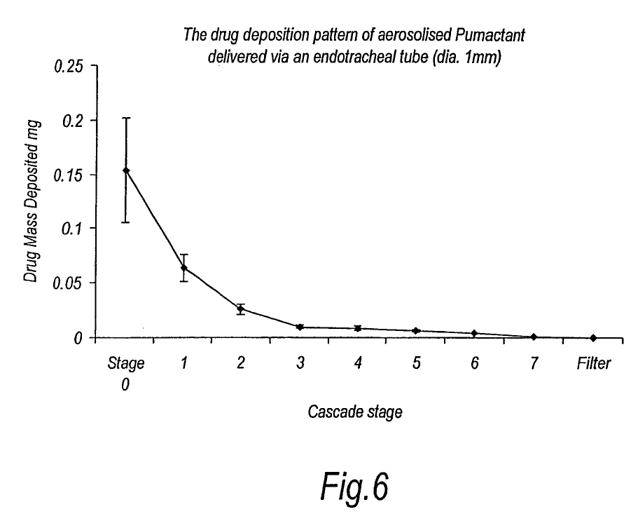

The deposition was measured in vitro using an Andersen cascade impactor. The results are given in FIG. 6.

The following results were obtained. (The initial baseline assessment 10 from tracheal washings are given in Table 1:

TABLE 1
BASELINE ASSESSMENT OF SUBJECT PRIOR TO STUDY START

| Macroscopic appearance | | Microscopic appearance | |
|---|---|---|---|
| Mucus | +++ | Neutrophils | +++ |
| Cloudy | trace | Deg Neutrophils | + |
| Blood | + | Macrophages | + |
| | | Siderophages | + |
| | | Epithelium | ++ |

General Inflammation score (0-12) 7

Wherein the following scoring severity was used:
−−−=non detected, +=mild, ++++=severe

The results obtained during the term of the study are illustrated in Table 2.

TABLE 2
TRACHEAL WASH DATA COLLECTED DURING THE
TERM OF THE STUDY

| | Nucleated | | | | |
|---|---|---|---|---|---|
| | Cells/1 | Neutro- | Mono- | Eosion- | Epi- |
| Date | Cell Type | phils | nuclear | phils | thelium |
| 19 Jan. 2002 | 0.3 H 10⁹ | −−− | −−− | −−− | −−− |
| (24 hours post | | | | | |
| treatment) | | | | | |
| 26 Jan. 2002 | 1.2 H 10⁹ | ++ | ++ | + | + |
| (Pre treatment) | | | | | |
| 26 Jan. 2002 | 0.8 H 10⁸ | + | ++ | −−− | ++ |
| 22 Feb. 2002 | HIGH | 28% | 24% | −−− | 48% |
| centrifuged deposit | | | | | |
| smear cell | | | | | |
| density(Pre | | | | | |
| treatment) | | | | | |
| 25 Feb. 2002 | LOW | 32% | 27% | 3% | 38% |
| centrifuged deposit | | | | | |
| smear cell density | | | | | |
| (Post treatment) | | | | | |

This Example shows the use of the device according to the invention in administering phospholipids in the treatment of equine respiratory disorders: Heaves in this instance. Primarily, the treatment is hypothesized to ‘work above the line’: to form a barrier over the epithelial surface it contacts with. The results from Table 2 indicate a reduction in epithelial shedding. When the epithelium is denuded or missing, the tissues below are exposed to insult, allowing the cascade of subsequent inflammatory mechanisms to proceed.

### EXAMPLE 2

The performance of an inhaler as shown in FIG. 1 was investigated using pumactant as a model drug. In particular, the influence of loaded dose on dry powder delivery and can pressure on aerosolization efficiency was investigated.

Reported clinical studies required a dosage regime of 4 H 100 mg, 8 hours and 30 mins prior to an allergen challenge [Babu, K S. et al, ibid]. Such high doses were well tolerated and early asthmatic response was abolished in all cases. However, due to pumactant's similarity to endogenous surfactant (e.g., low transition temperature and high moisture affinity), the energy required to aerosolize the powder was not achievable using conventional means.

Physical Characterization of Pumactant

Prior to in vitro testing, the micronized pumactant was first characterized for particle morphology, size distribution, moisture sorption and crystal structure.

The particle morphology of the micronized pumactant was investigated using scanning electron microscopy (SEM) (Jeol 6310: Jeol, Japan). Samples were mounted on carbon sticky tabs prior to analysis and gold coated (Edwards Sputter Coater, UK). Analysis of the data suggests discrete particulates with diameters less than 5 μm. Furthermore, the micronized particles appeared heavily agglomerated.

The particle-size distribution of the micronized pumactant was determined by laser light scattering (Mastersizer X, Malvern, UK), using a 100 mm lens and small volume stirring circulation cell. The micronized powder was dispersed in cyclohexane and ultrasonicated for 5 minutes prior to analysis (determined sufficient to fully de-aggregate the powder).

The median volumetric diameter (d0.5) for micronized pumactant was 1.49 μm±0.12 μm (n=3). Furthermore, the 10th and 90th percentile particle diameters were 0.81 μm±0.06 μm and 2.92 μm±0.31 μm, respectively suggesting the micronized drug to be of suitable size for inhalation therapy [Pritchard, J. N. 2001. The influence of lung deposition on clinical response. J. Aerosol Med. 14:S19-S26]. The particle size distribution appeared to be in good agreement with observations made by SEM.

In general, physical characterization of the pumactant suggests the potential of aerosolization would be relatively low. The powder has a micron size (<5 μm) and thus high surface area to mass ratio (cohesion). Furthermore, the material appeared heavily agglomerated, contained significant quantities of water and was predominately amorphous.

Moisture sorption profiles of the micronized Pumactant was conducted using dynamic vapour sorption (DVS) (DVS-1 Surface Measurement Systems, London, UK). Approximately 12 mg of powder was weighed into the sample pan of the DVS and subjected to a 0-90% relative humidity (RH) cycle (10% increments). Equilibration at each humidity was determined by a dm/dt of 0.0002% .min⁻¹.

The test results showed that initial water uptake at each specific humidity was very rapid (<30 mins) before stabilization. In general, an increase in mass of 14% was observed as humidity was increased from 0% RH to 90% RH. At 45% RH the percentage moisture content was approximately 6.2%. The subsequent in vitro studies were conducted at 45% RH (25° C.), and thus it would be reasonable to assume pumactant would be partially hydrated material.

Diffraction patterns for the micronized pumactant were obtained using X-ray powder diffraction (XRPD) using components and methods described elsewhere [Tobyn, M. J., McCarthy, G. P., Staniforth, J. N., Edge, S. 1998. Physicochemical comparison between microcrystalline cellulose and solidified microcrystalline cellulose. Int. J. Pharrn. 169:183-1 94].

Analysis of the XRPD diffractograph suggests a predominately amorphous material. Such observations are expected however, since the final two stages of pumactant production involves vacuum drying from an ethanol solution followed by cryo-micronization. It is interesting to note however, that a broad peak was observed at 21°2Θ, suggesting the presence of small semi-crystalline, or crystallite material in the powder.

Dispenser Device

The influence of loaded dose (20-250 mg) on delivery efficiency and can pressure (6-14 bar) on aerosolization efficiency (120 mg dose) was investigated. Pressurized canisters were filled with N₂ (O₂ free) (BOC, Manchester, UK), using a hand held pressurized filling machine (Manual Lab Plant, Pamasol, Switzerland), to 6, 8, 10, 12 and 14 bar (1 H10⁵ Pa) pressures. Filling pressures were checked against a calibrated pressure meter (Pamasol P700, Switzerland).

Delivered Dose Studies

The influence of loaded dose (0-250 mg) on the delivered dose (aerosolization of the powder bed) was investigated. Samples of pumactant were accurately weighed into pre-weighed sample vials, which were inserted into the device. Studies were conducted using 12 bar N₂ canisters. The device was actuated for a 10 second period into a fume hood. Delivered dose was calculated by mass difference. The device and actuator were cleaned using methanol and air-dried. All experiments were conducted at 45% RH and 25° C., and were randomized for loaded dose.

Aerosolization Efficiency Studies

The influence of can pressure on the aerosolization efficiency of 120 mg pumactant doses was investigated using the Marple Miller impactor (USP Apparatus 2) (Copley Instruments Ltd, Nottingham, UK). The Marple Miller impactor has five collection stages (in the form of sample cups), which at 60 L.min⁻¹ produce 5 effective aerodynamic cut-off diameters; 10 μm, 5 μm, 2.5 μm, 1.5 μm and 0.625 μm. In addition, a throat and after filter provide collection of particles >10 μm and <0.625 μm. A rotary vein pump (Gast, Buckinghamshire, UK) generated a flow rate of 60 L.min⁻¹ through the impactor, which was calibrated using a flow meter.

Approximately 120 mg of pumactant was weighed into a pre-weighed sample vial, which was inserted into the device. The actuator mouthpiece was inserted into a specially constructed mouthpiece and tested using the Marple Miller impactor at 60 L.min⁻¹ for 10 seconds. A 3 second delay prior to pressurized can actuation was instigated to allow equilibration of the pump. Drug concentrations in the sample vial, device and Marple Miller stages were calculated by mass difference using a 5-figure Sartorius balance. Data were processed to produce delivered dose (DD) (ex device), fine particle dose (FPD) (mass in stage 2 to filter) and fine particle fraction (FPF) (FPD/DD H 100). The FPD and FPF refer to deposited drug with an aerodynamic mass median diameter of less than <5 μm. The Marple Miller sample cups, filter stage throat and device were cleaned with methanol and air-dried between experiments.

As with the delivered dose studies, environmental conditions were 45% RH and 25° C. Experiments were randomized for can pressure.

Pumactant Aerosolization Efficiency

The efficiency of the device in delivering micronized pumactant was investigated. Initially the relationship between loaded dose and delivered dose (0-250 mg) was studied (12 bar canister pressure). Secondly, the aerosolization efficiency of the micronized pumactant (i.e., particles that would potentially be respirable (<5 μm)) was investigated as a function of canister pressure (6-14 bar). In this case a 120 mg loaded dose was choosen for similarity to clinical trial doses reported previously.

Delivered Dose Studies

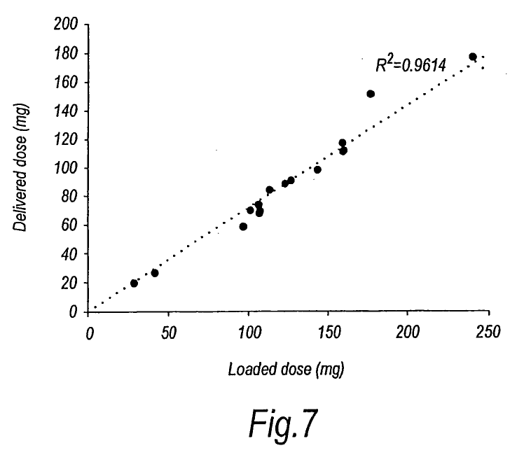

The relationship between loaded and delivered dose is represented graphically in FIG. 7. In general, a linear relationship (R²=0.96) between loaded and delivered dose was observed (n=18). Device efficiency across all doses was 70.1%±6.3% (n=18). As expected, no correlation between loaded dose and device efficiency was. found (Pearson analysis).

Influence of Canister Pressure on Fine Particle Aerosolization

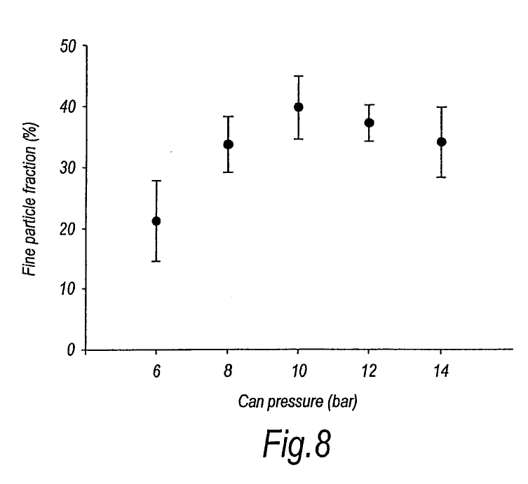

The influence of can pressure on the aerosolization efficiency of the PADD device, using a Marple Miller impactor, is summarized in the Table 3 and illustrated in FIG. 8.

TABLE 3
INFLUENCE OF CAN PRESSURE ON AEROSOLISATION
EFFICIENCY

| | | | Fine particle | Fine particle |
|---|---|---|---|---|
| Pressure | Loaded dose, | Delivered dose, | dose,² | fraction,³ |
| (bars¹) | (mg ± sd) | (mg ± sd) | (mg ± sd) | (% ± sd) |
| 6 | 120.7 ± 1.5 | 35.7 ± 8.8 | 7.5 ± 2.7 | 21.1 ± 6.6 |
| 8 | 118.4 ± 6.6 | 79.3 ± 10.1 | 27.0 ± 7.1 | 33.7 ± 4.6 |
| 10 | 116.8 ± 1.4 | 79.2 ± 7.7 | 31.4 ± 5.1 | 39.7 ± 5.2 |
| 12 | 121.0 ± 7.0 | 86.4 ± 2.8 | 32.1 ± 3.0 | 37.2 ± 3.0 |
| 14 | 120.4 ± 0.9 | 86.8 ± 6.5 | 29.3 ± 3.0 | 34.0 ± 5.8 |

¹1 bar = 1 H 10⁵ Pa,
²Deposited fraction collected from stage 2-filter (<5 μm),
³Percentage fraction below 5 μm

The mean loaded dose throughout the study was 119.5±4.1 mg. Statistical analysis (ANOVA, Fisher pair wise, p<0.05) indicated no significant variance between loaded doses and canister pressure studied.

Statistical analysis of delivered dose (ANOVA, p<0.05) indicated canister pressure had significant influence on powder bed fluidization. However, Fisher's pair-wise analysis indicated this to only be the case between 6 and 8 bars (35.7 mg±8.8 mg at 6 bar to 79.3 mg±10.1 mg at 8 bar). Thus, it is reasonable to suggest that the device could be successfully used between 8 and 14 bars.

Although delivered dose is a good estimation of the powder bed fluidization efficiency, it is not indicative of the aerosolization efficiency of the system (that is to say, the efficiency of the system in de-agglomerating the micronized powder agglomerates). The fine particle dose therefore is used to describe the potential dose that would be received in the lower respiratory tract (lower bronchiole) [Pritchard supra].

Previous investigations using micronized pumactant (˜50 mg) and a commercial dry powder inhaler (Cyclohaler®, Novartis, Surrey, UK), showed comparable delivered dose values to the present device, but resulted in no FPD [Young, P. M., Thompson, J., Price, R., Woodcock, D., Davies, K. 2003. The use of a novel hand held device to deliver high respirable fractions of high dose dry powder active agents to the lung. J. Aerosol Med. 16:1921. Such observations suggested the energy of the Cyclohaler® was not sufficient to de-agglomerate the powder once entrained in an air stream. In comparison, the mean FPD using the present device and 6 bar canister was 7.5±2.7 mg (n=3). This rose significantly (Fisher's pair-wise, p<0.05) to 27.0 mg±7.1 mg at 8 bar (n=3). Further increases in canister pressure did not result in significant changes in FPD. However, it is interesting to note a decrease in the standard deviation was observed as pressure was increased (with a FPD of 29.3 mg±3.0 mg being observed at 14 bar (n=)).

Comparison of the FPF indicated similar findings to the FPD, with a significant increase (Fisher pair-wise, p<0.05) in FPF between 6 and 8 bar canister pressures (21.1 mg±6.6 mg and 33.7 mg±4.6 mg at 6 and 8 bars, respectively). However, the relative difference between 6 and 8 bar FPF values when compared with FPD was less. Such observations are most likely attributed to the relative differences in delivered doses between the two pressures. Again, no significant difference (ANOVA, Fisher pair-wise, p<0.05) in the FPF for tests conducted between 8-14 bar pressurized canisters were observed. A mean FPF of 36.1 mg±4.8 mg was observed across the range: 8-14 bar.

Initial studies using the pressurized aerosol dry-powder delivery device according to the invention show that the aerosolization of micronized pumactant is possible over the range 20-250 mg. Furthermore, in vitro studies of 120 mg loaded doses indicated fine particle fractions of >30 weight % (˜30 mg FPD) when delivered using 8-14 bar aerosolization pressures. Although previous studies have demonstrated the delivery of high dose medicaments is possible, the combination of active device design and carrier free formulation enables high-energy powder aerosolization while circumventing issues that may arise with the use of high dose excipients.

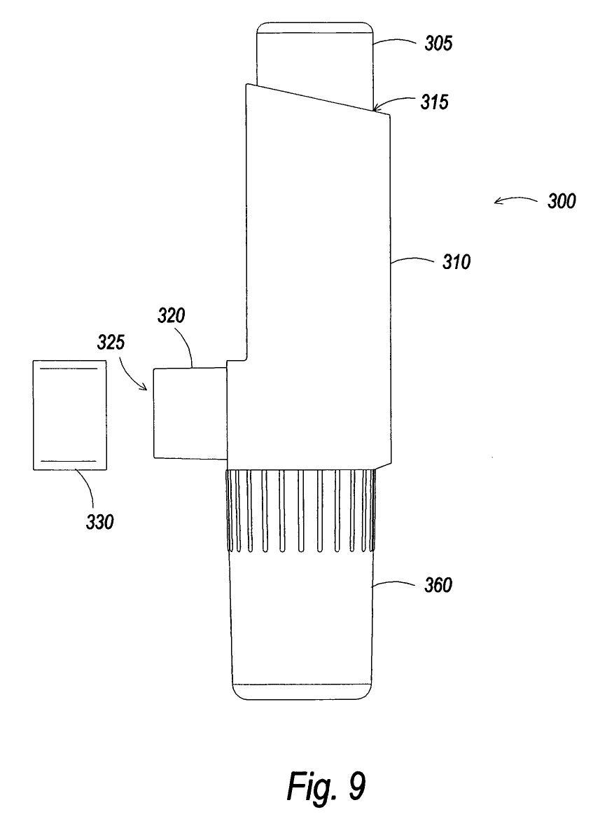

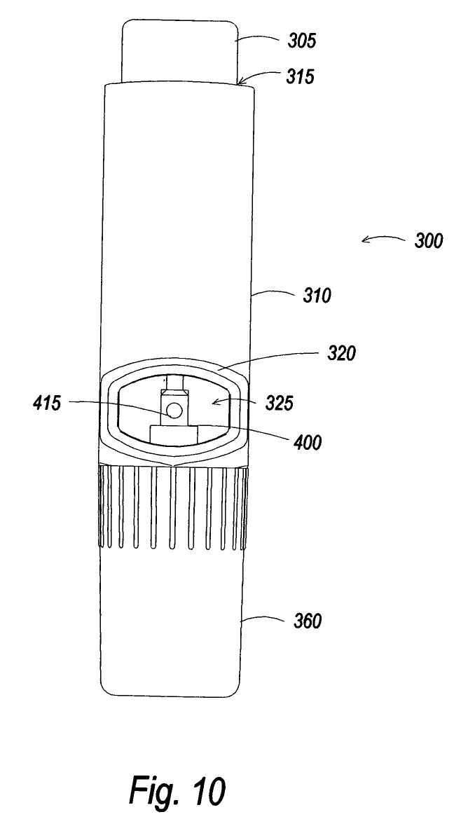

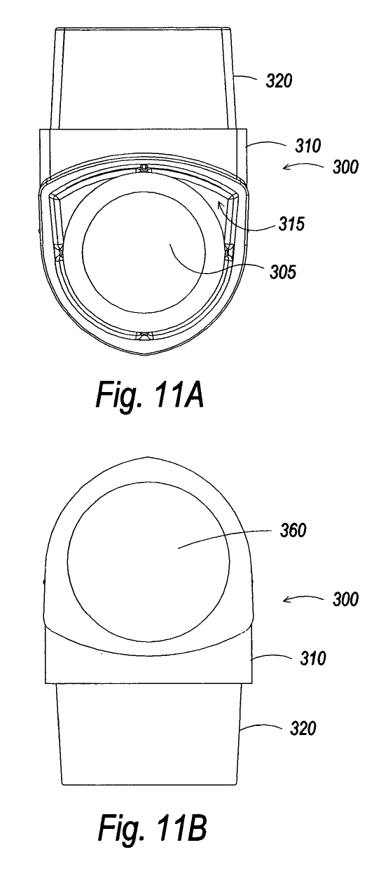

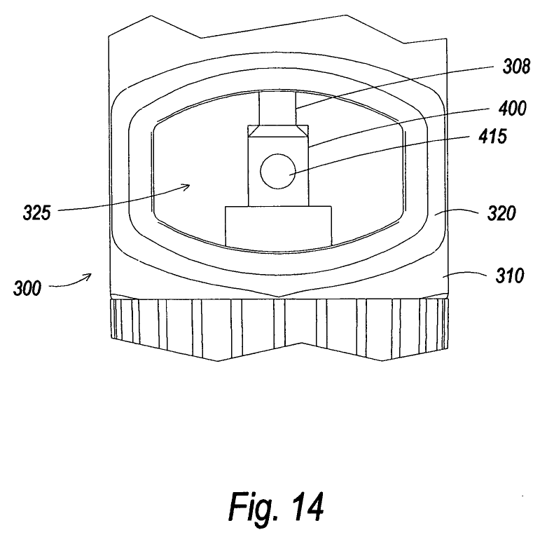

FIGS. 9-15 show a fourth embodiment of a device 300 according to the invention. Like reference numerals are used to represent like features of the first embodiment. The device 300 delivers a medicament in a dry powder form in larger doses than prior devices can achieve. Many prior devices cannot deliver dry powder or can only effectively deliver dry powder in a minimal amount. The device 300 provides a delivered dose of a dry powder medicament of up to approximately 40% to approximately 50% by weight of a loaded dose. One of ordinary skill in the art will recognize that higher percentages of the delivered dose, such as 55%, 65%, 75%, or 85%, and lower percentages of the delivered dose, such as 10%, 20%, or 30%, may be achieved depending upon the pressure of the propellant gas, the amount of loaded dose, etc. The device 300 is capable of delivering an aerosol of respirable (having a mass median aerodynamic diameter of less than 5 microns) medicament particles. The respirable particles may have a mass median aerodynamic diameter of less than 5 microns, less than 4 microns, less than 3 microns, less than 2 microns, or less than 1 micron. Other larger medicament particles may be delivered by the device 300, such as those having a mass median aerodynamic diameter of less than 20 microns or less than 10 microns.

The device 300 positions a power source 305 and the receptacle 54 in the same axis. The power source 305 provides the propellant gas for aerosolizing the medicament. The power source 305 may be a canister, cylinder, container or other source of the propellant gas. The power source 305 may be connected to or inserted into the device 300. The specific propellant gas may be carbon dioxide, nitrogen, argon, helium, air, fluorocarbons such as HFA-134a, or other compressed gases which can be delivered to the body. A canister of nitrogen gas at a pressure of approximately 6 bar to approximately 20 bar is a preferred power source 305. One of ordinary skill in the art will recognize that canisters of nitrogen gas at, for example, 8 bar, 10 bar, 12 bar, 14 bar, 16 bar, 18 bar, etc. may be used.

The device 300 includes a main body 310, which may be constructed from a pharmaceutical grade molded plastic. The main body 310 provides a housing for the device 300. The main body 310 receives the power source 305 through a top opening 315 of the main body 310. The main body receives the receptacle 54 opposite of the power source 305, i.e., on the bottom of the main body 310 shown as a bulkhead 350. This arrangement provides a direct, non-turning path for the propellant gas to enter the main body 310 and pass through the main body 310 to the receptacle 54. The direct, non-turning path includes a venturi, but the general direction of the propellant gas is not changed as it passes through the main body 310. The bulkhead 350 acts as an interface for the receptacle 54. The terms “top” and “bottom” are used for reference purposes only, as the invention may be practiced in other orientations, such as in a horizontal fashion. This embodiment of the invention functions with or without the inlet tube 63 described in other embodiments of the invention.

The device 300 includes an ergonomic design, well suited for the user to self-administer the aerosol. A user applies a direct force to the power source 305 and the main body 310 to deliver the aerosol. A mouthpiece 320 generally protrudes from the main body 310. In the embodiment shown in FIGS. 9-15, the mouthpiece 320 protrudes in a generally perpendicular manner, although other embodiments of the invention may include a mouthpiece protruding at other angles.

The mouthpiece 320 forms a mouthpiece opening 325. In the embodiment shown, the mouthpiece 320 is integral with main body 310. The mouthpiece 320 defines an open passage to deliver the aerosol from the main body 310 to the user. The mouthpiece 320 delivers the medicament in the form of the aerosol to the user. A dust cap 330 may be used to cover the mouthpiece opening 325.

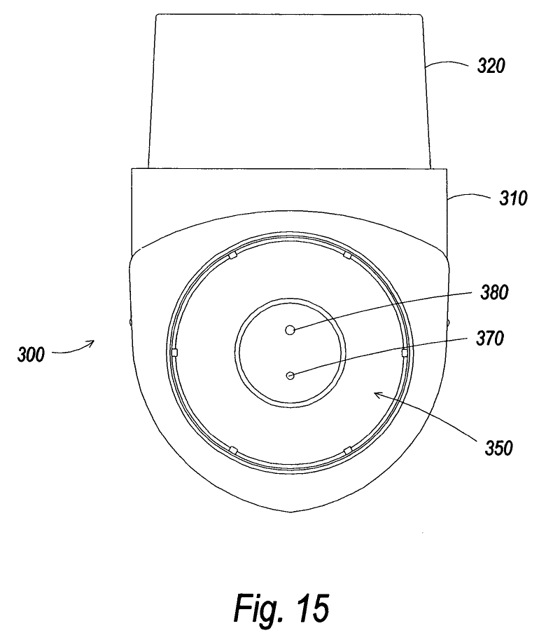

The bulkhead 350 receives the receptacle cover 360. The receptacle cover 360 contains the receptacle 54, and the receptacle cover 360 is threadably received by the bulkhead 350. Thus, securing the receptacle cover 360 to the bulkhead 350 connects the receptacle 54 to the bulkhead 350. The bulkhead 350 is shown in FIG. 15 with the receptacle 54 and the receptacle cover 360 removed.

A gasket 365 is positioned between the receptacle cover 360 and the bulkhead 350. As the bulkhead 350 threadably receives the receptacle cover 360, the gasket 365 is compressed between the receptacle cover 360 and the bulkhead 350, thus sealing the receptacle 54 against the bulkhead 350. The gasket 365 and the receptacle 54 may be replaced with a screw top vial that is threadably received by the bulkhead 350. This arrangement eliminates the need for the gasket 365, which may make the device 300 more desirable to users.

The bulkhead 350 includes an outlet 370 and an inlet 380. The outlet 370 is in open communication with the mouthpiece opening 325 and the receptacle 54 containing the medicament. The inlet 380 is in open communication with the power source 305 and the receptacle 54 containing the medicament. The receptacle 54 contains the medicament in a powder form. The propellant gas from the power source 305 enters the receptacle 54 via the inlet 380. The propellant gas then aerosolizes the medicament contained in the receptacle 54 forming an aerosol of medicament in the receptacle 54 that is propelled into the mouthpiece 320 via the outlet 370 for inhalation by the user.

The receptacle 54 may contain approximately 20 mg to approximately 250 mg of medicament. One of ordinary skill in the art will recognize that the receptacle 54 may contain, for example, 40 mg, 80 mg, 120 mg, 160 mg, 200 mg, etc. of medicament.

The receptacle 54 may have a total volume of approximately 1 ml to approximately 10 ml. One of ordinary skill in the art will recognize that the receptacle 54 may have a total volume of, for example, 2 ml, 4 ml, 6 ml, 8 ml, etc. The receptacle 54 may be a glass, plastic, or other container suitable to hold a dry powder pharmaceutical. The receptacle 54 may also be replaced with a blister pak having a total volume of 1 ml or less.

The diameter of the outlet 370 may range from approximately 0.5 mm to approximately 2.5 mm. One of ordinary skill in the art will recognize that the diameter of the outlet 370 may be, for example, 0.8 mm, 1.1 mm, 1.7 mm, 2.0 mm, etc. A preferred diameter for the outlet 370 is approximately 1.4 mm. The diameter of the outlet 370 may be varied to best accommodate the physical/chemical characteristics of the particular medicament to be aerosolized.

The diameter of the inlet 380 may range from approximately 0.6 mm to approximately 1.8 mm. One of ordinary skill in the art will recognize that the diameter of the inlet 380 may be, for example, 0.8 mm, 1.0 mm, 1.4 mm, 1.6 mm, etc. A preferred diameter of the inlet 380 is approximately 1.2 mm. The diameter of the inlet 380 may be varied to best accommodate the physical/chemical characteristics of the particular medicament to be aerosolized.

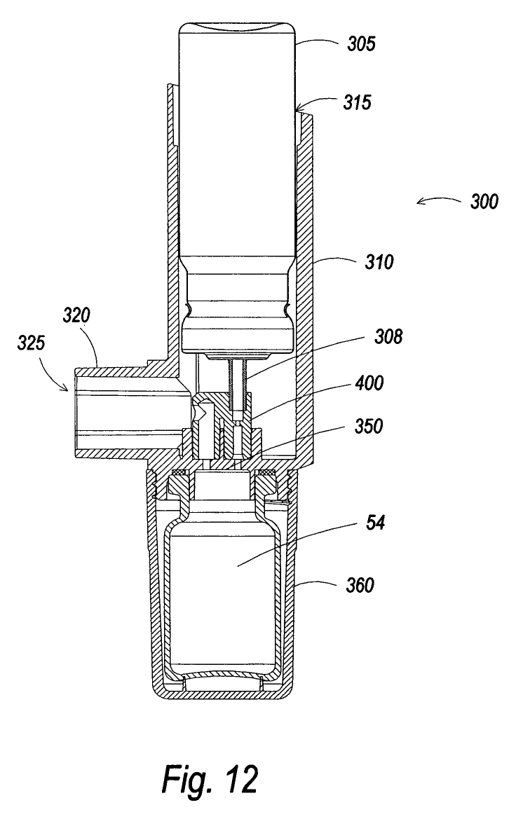

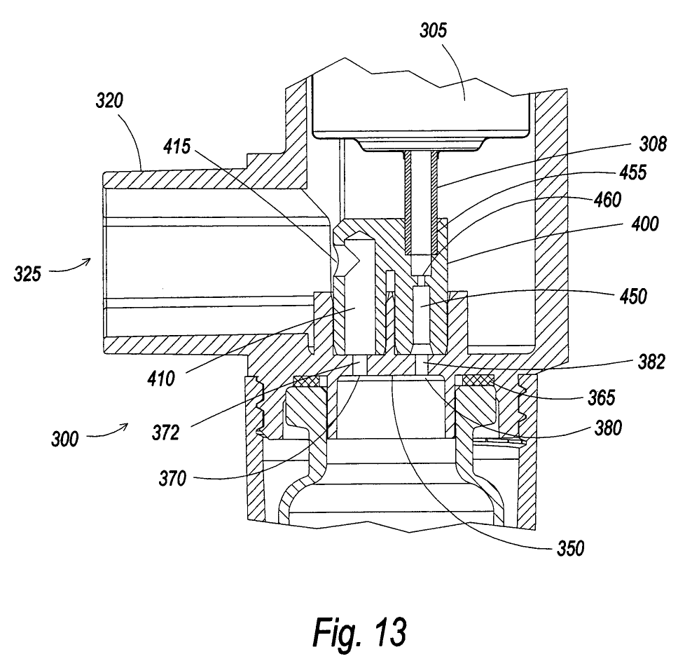

With reference to FIGS. 12 and 13, the interior portions of the device 300 will now be described. The main body 310 fixedly receives a stem block 400. In other embodiments, the stem block 400 may be integral with the main body 310.

As shown in FIG. 13, the stem block 400 includes an outlet path 410 that is in open communication with the mouthpiece 320 and the outlet 370. The stem block 400 further includes an inlet path 450 that is in open communication with the inlet 380 and a propellant opening 455. The propellant opening 455 is bored or formed in the stemblock 400 and provides an entrance for the propellant gas into the inlet path 450 of the stem block 400. The propellant opening 455 receives a power source inlet 308 from the power source 305. In the embodiment shown in FIGS. 9-15, the power source inlet 308 is a tube or conduit extending from the power source 305.

The inlet path 450 and the outlet path 410 may be bored through the stem block 400 or may be formed during the molding of the stem block 400. In this embodiment, the stem block 400 positions the inlet path 450 and the outlet path 410 in a generally parallel arrangement.

The propellant opening 455 receives the power source inlet 308 from the power source 305. The propellant gas next passes through an inlet venturi 460. The inlet venturi 460 decelerates the flow of the propellant gas into the inlet path 450. By decelerating the propellant gas, a better aerosolization of the medicament is achieved. The inlet venturi 460 may be formed or bored into the stem block 400. An average diameter of the inlet venturi 460 may range from approximately 0.3 mm to approximately 0.9 mm. One of ordinary skill in the art will recognize that the average diameter of the inlet venturi 460 may be, for example, 0.4 mm, 0.6 mm, 0.8 mm, etc. A preferred average diameter for the inlet venturi 460 is approximately 0.7 mm. The average diameter of the inlet venturi 460 may be varied to best accommodate the physical/chemical characteristics of the particular medicament to be aerosolized.

The inlet venturi 460 may be positioned approximately 5 mm to approximately 20 mm from the bulkhead 350. One of ordinary skill in the art will recognize that the inlet venturi 460 may be positioned, for example, 8 mm, 11 mm, 14 mm, 17 mm, etc. from the bulkhead 350. This distance between the inlet venturi 460 and the bulkhead 350 also assists in decelerating the propellant gas to achieve better aerosolization of the medicament.

The size and positioning of the inlet venturi 460, the outlet 370, the inlet 380 are important in delivering the aerosolized medicament and the high dose of delivery of the medicament. The inlet venturi 460 and the inlet 380 regulate the propellant gas entering the receptacle 54 such that the propellant gas aerosolizes the medicament. The outlet 370 regulates the flow of the aerosol out of the receptacle 54.

The bulkhead 350 includes an outlet passage 372 and an inlet passage 382 formed or bored through the bulkhead 350 to provide for the propellant gas and the aerosol to pass through the bulkhead 350. The outlet passage 372 openly connects the outlet 370 with the outlet path 410. The inlet passage 382 openly connects the inlet 380 with the inlet path 450. As can be ascertained from the Figures, the bulkhead positions the inlet 380 to direct the incoming propellant gas toward the bottom of the receptacle 54 and the medicament therein. The medicament is then aerosolized, and exits via the outlet 370. In order to increase the aerosolization of the medicament, the outlet 370 is positioned away from the medicament.

With continued reference to FIG. 13, the outlet 370 opens to the outlet passage 372 in the bulkhead 350, the outlet passage 372 opens to the outlet path 410 in the stemblock 400, and the outlet path 410 opens into the mouthpiece 320. In this embodiment, the outlet path 410 includes an opening 415 in open communication with the mouthpiece 320. The flow of the aerosol through the mouthpiece 320 is generally perpendicular to the flow of the aerosol through the outlet path 410 in this embodiment.

Importantly, the device provides for the delivery of an aerosol of a dry powder. A dry powder includes a substance containing less than or equal to 25% water by weight. Preferably, the dry powders have less than or equal to 15% water by weight. Certain dry powders have less than or equal to 1%, 2%, 3%, 4% or 5% water by weight.

### EXAMPLE 3

The device 300 was studied using a dry powder medicament called Zofac™. Notably, up to approximately 40 mg of Zofac™ was delivered in an aerosol with a single actuation from an a loaded dose of approximately 100 mg of Zofac™. Zofac™ is composed of two synthetic phospholipids, dipalmitoylphosphatidylcholine (DPPC) and unsaturated phosphatidylglycerol (PG), in a ratio of 7:3. Zofac™ has mass median aerodynamic diameter of the less than 5 microns. Zofac™ contains not more than 4% by weight of water. Of course, other dry power medicaments may be used with the device 300.

The characteristics of the aerosols delivered by the device 300 were evaluated by both Malvern laser diffraction and Anderson cascade impactor studies. The power source 305 was a nitrogen canister at 10 bar or 14 bar. The amount of Zofac™ delivered at 10 bar and at 14 bar from a 100 mg loaded dose is shown in Table 4. A higher delivered dose was observed with 14 bar compared to 10 bar pressure in the nitrogen canisters. Device efficiency (% respirable dose) under these conditions was 18-22% at 10 bar and 19-31% at 14 bar when evaluated by Malvern and Anderson cascade impactor methods. Delivered dose ranged from 30-44%.

TABLE 4
DELIVERY OF ZOFAC ™

| | Malvern | | | | Anderson | | | |
|---|---|---|---|---|---|---|---|---|
| | Laser Diffraction² | | | | Cascade Impactor⁴ | | | |
| Parameter | 10 bar | sd³ | 14 bar | Sd | 10 bar | sd | 14 bar | sd |
| Loaded | 99.6 | 2.0 | 101.3 | 2.1 | 100.6 | 2.2 | 99.9 | 2.8 |
| Dose (mg) | | | | | | | | |
| Delivered | 29.9 | 6.7 | 44.1 | 7.7 | 38.9 | 2.1 | 44.3 | 1.0 |
| Dose (mg) | | | | | | | | |
| Fine Particle | 21.8 | 4.4 | 31.6 | 6.0 | 18.0 | 0.1 | 19.1 | 2.0 |
| Dose (mg) | | | | | | | | |
| Delivered | 30.0 | 7.0 | 43.5 | 7.6 | 38.6 | 1.2 | 44.4 | 1.9 |
| Dose (%) | | | | | | | | |
| Fine Particle | 73.6 | 5.7 | 71.7 | 6.3 | 46.5 | 2.9 | 43.1 | 5.3 |
| Fraction (%) | | | | | | | | |
| Device | 21.9 | 4.7 | 31.1 | 5.8 | 17.9 | 0.5 | 19.1 | 1.6 |
| Efficiency (%)¹ | | | | | | | | |

¹Device Efficiency = Delivered Dose (mg) × Fine Particle Fraction/Loaded Dose (mg)
²n = 15
³sd = standard deviation
⁴n = 3

The measurement of the delivered dose was determined gravimetrically. The weight of the device 300 and the loaded receptacle 54 was recorded before and after firing the device 300, with the difference being the delivered dose.

A Malvern Spraytec system was used to perform the laser diffraction to measure particle size distribution for determination of the respirable fraction (<5 μm). The device 300 was positioned with the mouthpiece 320 being 5 cm from the laser beam, such that the plume was fired horizontally and the laser beam intersected the direction of plume travel at 90 degrees. Visually, the laser sampled the center of the plume in the vertical direction. Measurements performed were Dv(10), Dv(50), Dv(90), % Transmittance and % Volume<5 μm.

To support the Malvern results, particle size distribution was also measured by gravimetric methods with an Anderson cascade impactor. Flow rate was set at 90 L/min to deliver a volume of 4 L (2.7 sec).

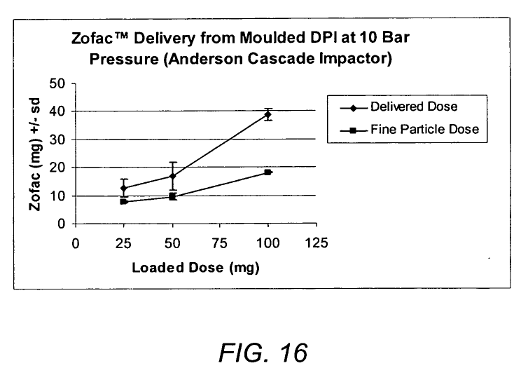

The effect of loaded dose on delivery characteristics as measured by the Anderson cascade impactor is shown in FIG. 16. The moulded DPI referred to in FIG. 16 is the device 300. Delivered dose and fine particle fraction are shown in mg.

These studies show that the device 300 provides for the delivery of more than 40 mg Zofac™ from a single actuation of a 100 mg dose. The device 300 has an efficiency in the range of 18-31%, based on respirable fraction. The device 300 provided a higher percent delivered dose with the higher pressure in the nitrogen canisters. The device 300 is a useful delivery device for patients who have low inspiratory flow and for applications requiring delivery of large doses of drugs.

As stated previously, each embodiment of the device may be used with a dry powder for the treatment and/or relief of respiratory diseases or conditions. For example, the device may be used for the treatment or relief of all types of asthma, including allergic asthma, perennial asthma, environmental asthma, exercise-induced asthma, cold-induced asthma, chemical-induced asthma, mild asthma, mild to moderate asthma, severe asthma, as well as other diseases and conditions, such as acute respiratory distress syndrome, age-related loss of endogenous surfactant, Baker's lung, bronchiectasis, acute and chronic bronchitis, non-allergenic bronchitis, bronchospasm, allergen-induced bronchospasm, cold-induced bronchospasm, exercise-induced bronchospasm, chronic obstructive pulmonary disease (COPD), cystic fibrosis, emphysema, HIV induced pulmonary complications, idiopathic pulmonary fibrosis, nasal congestion, nasal rhinitis due to allergens or rhinoviruses, otitis, otitis media, serous otitis, pneumonia, sarcoidosis, silicosis, sinusitis, chronic sinusitis, asbestosis, black lung, and secondary lung infections from rhinoviruses. The device may also be used for the treatment of pulmonary damage from a wide variety of causes, including but not limited to, damage from inhalation of particulates, such as silicates, asbestos, carbon and coal, or from inhalation of gases such as superheated air, smoke, hyperbaric oxygen, or toxic gases or fumes, such as hydrogen sulfide, gasoline, turpentine, chloroform, carbon tetrachloride, formaldehyde, dry cleaning solvents, paint solvents, and aldehydes.

It is also expected that the device may be useful in the delivery of therapeutic substances such as mucosally administered antigens, antibiotics, vaccines, gene therapies, recombinant DNA, proteins, peptides, and cromolyn sodium which can be administered as a dry powder alone or in combination with other dry powders that may be aerosolized by the device. It is envisioned that certain of these delivered substances will be part of the treatment or diagnosis of diseases and conditions unrelated to the pulmonary system. Further, the device may be useful in the deliver of radiolabels, luminescent and non-radiolabeled markers, vitamins, strontium, and other compounds that may act as tracers (i.e. markers that are mixed with a material to follow the material within its physical or biological matrix).

As is evident from the foregoing description, certain aspects of the present invention are not limited by the particular details of the examples illustrated herein, and it is therefore contemplated that other modifications and applications, or equivalents thereof, will occur to those skilled in the art. It is accordingly intended that the claims shall cover all such modifications and applications that do not depart from the spirit and scope of the present invention.

1. A delivery device for a medicament, comprising:
a housing,
a receptacle containing a medicament in the form of a powder,
a power source comprising a propellant gas,
the housing comprises an inlet and an outlet for the receptacle,
a stem block comprising an outlet path and an inlet path, the outlet path is in open communication with the outlet, and the inlet path is in open communication with the inlet, a venturi, and a propellant gas opening, and
the inlet directs the propellant toward the medicament to aerolize the medicament, and the outlet is positioned away from the medicament.

2. The device according to claim 1, wherein the receptacle is removable from the housing.

3. The device according to claim 1, wherein the power source is removable from the housing.

4. The device according to claim 1, wherein the venturi decelerates the propellant gas.

5. The device according to claim 1, wherein the outlet path is in open communication with a mouthpiece.

6. The device according to claim 1, wherein the propellant gas opening provides an entrance for the propellant gas into the inlet path.

7. The device according to claim 1, wherein the inlet path and the outlet path are bored through the stem block or formed during a molding of the stem block, wherein the stem block positions the inlet path and the outlet path in a generally parallel arrangement.

8. The device according to claim 1, wherein the device comprises a bulkhead that interfaces with the receptacle.

9. The device according to claim 8, wherein the bulkhead comprises an outlet passage and an inlet passage formed or bored through the bulkhead to allow for the propellant gas and an aerosolized medicament to pass through the bulkhead.

10. The device according to claim 9, wherein the outlet path is in open communication with a mouthpiece and the outlet, wherein the outlet passage openly connects the outlet with the outlet path, and the inlet passage openly connects the inlet with the inlet path.

11. The device according to claim 1, wherein the receptacle has a bottom containing the medicament, and the receptacle has a top connecting to the housing, and the outlet is arranged to open into the receptacle at the top of the receptacle.

12. The device according to claim 11, wherein the outlet opens into the receptacle, and a receptacle cover positions the receptacle.

13. The device according to claim 1, wherein the device positions the power source and the receptacle in the same axis.

14. The device according to claim 1, wherein the outlet does not extend into the receptacle.

15. The device according to claim 1, wherein the outlet is formed as a hole in the housing which is in open communication with the outlet path in the housing which connects to the exterior of the housing.

16. The device according to claim 1, wherein the outlet regulates the flow of the aerosolized medicament out of the receptacle.

17. The device according to claim 1, wherein the device provides a delivered dose of up to approximately 40% to approximately 50% by weight of a loaded dose.

18. The device according to claim 1, wherein the outlet is in open communication with an outlet path which connects to the exterior of the device.

19. The device according to claim 1, wherein the powder is a dry powder containing less than or equal to 25% by weight water.

20. The device according to claim 1, wherein the device is a handheld device.

21. The device according to claim 1, wherein the power source is a canister of gas.

22. The device according to claim 1, wherein the power source has a valve, and the valve is actuated by the user to deliver the aerosol of the medicament.

23. The device according to claim 1, wherein the device delivers the aerosolized medicament powder, including a medicament powder having a mass median aerodynamic diameter of less than 5 microns.

24. The device according to claim 1, wherein the outlet opens into a screw top vial.

25. The device according to claim 1, wherein a stem block comprises the outlet path and the inlet path, wherein the stemblock forms the venturi.

26. The device according to claim 1, wherein the inlet and the venturi regulate the propellant gas entering the receptacle.

27. A kit, comprising: the delivery device according to claim 1, a canister of a gas as the power source, a receptacle containing a medicament in powder form, and a receptacle cover.

28. A delivery device for a medicament, comprising:
a housing,
a receptacle containing a medicament in the form of a powder,
a power source comprising a propellant gas, wherein the housing receives the receptacle opposite of the power source,
an inlet path that provides a direct path for the propellant gas to reach the receptacle,
the housing comprises an inlet and an outlet for the receptacle, the inlet being in fluidic communication with the inlet path, and
the inlet directs the propellant toward the medicament, and the outlet is positioned away from the medicament.

29. A method of dispensing a medicament as an aerosol to a patient, comprising:
providing a receptacle having an opening, the receptacle containing a medicament in powder form;
connecting the receptacle to a housing, the housing having a stem block comprising an outlet path and an inlet path, the outlet path is in open communication with an outlet, the inlet path is in open communication with an inlet, a venturi, and a propellant gas opening,
discharging a propellant gas into the propellant gas opening and through the venturi to the inlet path, the inlet path directing the propellant gas through the inlet towards the medicament, the medicament being spaced from the medicament; and
forming an aerosol by transfer of energy from the propellant to the medicament; and discharging the aerosol through the outlet of the housing provided at the opening of the receptacle.

30. A delivery device for a dry powder medicament, comprising:
a delivery device that directs a propellant gas toward a dry powder medicament in a receptacle to aerosolize the dry powder medicament,
the delivery device positions an outlet away from the medicament in the receptacle to provide for the aerosolization of the dry powder medicament in the receptacle, and
the aerosolization passes through the outlet for delivery.

31. The delivery device according to claim 30, wherein the delivery device decelerates the propellant gas before the propellant gas aerosolizes the dry powder medicament.

32. The delivery device according to claim 30, wherein the delivery device decelerates the delivery of the aerosolization of the dry powder medicament.

33. The delivery device according to claim 30, wherein the delivery device comprises an inlet that directs the propellant gas toward the dry powder medicament.

34. The delivery device according to claim 30, wherein the dry powder medicament in the receptacle has a mass median aerodynamic diameter of less than 20 microns.

35. The delivery device according to claim 30, wherein the receptacle has a bottom containing the medicament and a top which connects to the delivery device, and the outlet is arranged to open into the receptacle unit at the top of the receptacle unit.

36. The delivery device according to claim 30, wherein a stable aerosol of the medicament is formed upon activation of the device.
